(19) United States
(12) Patent Application Publication
(10) Pub. No.: US 2006/0293549 A1
(43) Pub. Date: Dec. 28, 2006

(54) Method for the double-bond isomerisation of olefins

(75) Inventors: Marcus Sigl, Mannheim (DE); Ulrich Steinbrenner, Neustadt (DE)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/556,462

(22) PCT Filed: May 6, 2004
(86) PCT No.: PCT/EP04/04818

§ 371 Date: Nov. 10, 2005

(30) Foreign Application Priority Data
May 14, 2003 (DE) 103215239

(51) Int. Cl. C07C 5/25

(52) U.S. Cl. 585/670

(57) ABSTRACT

A process for preparing a C₄- to C₁₂-olefin (olefin A) from another C₄- to C₁₂-olefin (olefin B), wherein olefin (A) and olefin (B) differ with regard to the position of the double bond, and wherein a gaseous mixture comprising olefin (B) and from 0.01 to 10% by weight, based on the total amount of hydrocarbon compounds in this mixture, of a compound having a dipole moment of from 0.5 to 5 debye (compound P) is contacted with a basic catalyst at a temperature of from 200 to 700° C.

The present invention relates to a process for preparing a C₄- to C₁₂-olefin (olefin A) from another C₄- to C₁₂-olefin (olefin B), wherein olefin (A) and olefin (B) differ with regard to the position of the double bond, and wherein a gaseous mixture comprising olefin (B) and from 0.01 to 10% by weight, based on the total amount of hydrocarbon compounds in this mixture, of a compound having a dipole moment of from 0.5 to 5 debye (compound P) is contacted with a basic catalyst at a temperature of from 200 to 700° C.

C₄- to C₁₂-olefins, for example butenes, are important starting compounds for preparing compounds having relatively high added values. They are prepared, for example, in steam crackers by cracking naphtha. However, the hydrocarbon mixture formed in the steam cracker often does not correspond to the demand for the individual hydrocarbons. This is also true of the double bond isomers of butene. Large amounts of 1-butene are required, for example, to prepare 3-hexene therefrom by metathesis or by hydroformylation of C₅-aldehydes. Processes are therefore required by which the individual double bond isomers can be interconverted.

It is common knowledge that the isomerization of 2-butenes to 1-butene is an equilibrium reaction. cis-2-Butene, trans-2-butene and 1-butene are in equilibrium with each other. The thermodynamic data are listed in D. Stull, “The Chemical Thermodynamics of Organic Compounds”, J. Wiley, New York 1969.

In this text, “isobutene” is not included under “butenes”.

WO 02/094433 describes a process for preparing 1-butene from 2-butenes, in which the catalysts used are magnesium oxide, calcium oxide, barium oxide, lithium oxide or mixtures thereof. However, it is explicitly recommended (cf. p. 7) to remove polar compounds such as water and alcohol from the feedstock.

U.S. Pat. No. 4,217,244 likewise describes a process for preparing 1-butene from 2-butenes over a magnesium oxide catalyst. It is recommended here too to free the feedstock of moisture by treating with molecular sieve (cf. p. 4, lines 22 ff).

In Catalysis Surveys from Japan, Vol. 5, No. 2, April 2002, pages 81 ff, T. Yamaguchi et. al. describe the double bond isomerization in olefins over alkali metal oxide catalysts on aluminum oxide or zirconium dioxide supports. To prepare the catalysts, the supports are initially impregnated with solutions of nitrates or carbonates of the alkali metals. Subsequently, the impregnated supports are heated to temperatures above the decomposition temperature of the nitrates or carbonates, and the alkali metal oxides are formed. HU-B-204021 discloses a process for preparing 1-butene from 2-butenes over an alkali metal oxide on an aluminum oxide support.

It is an object of the present invention to provide a process by which double bond isomers of olefins can be interconverted with high selectivity. It is a further object to configure the process in such a way that the on-stream times of the basic catalysts used, which are known to be short at the high temperatures (200- 500° C.), are prolonged. In particular, the invention relates to a process by which the 1-butene fraction in C₄ hydrocarbon streams can be increased at the expense of the 2-butenes fraction.

We have found that this object is achieved by the invention defined at the outset.

The process according to the invention is of particular significance when the olefin (B) used is cis-2-butene, trans-2-butene, 1-butene or mixtures thereof. Usually, the butenes are in the form of a mixture with other hydrocarbons such as n-butane, isobutane or isobutene. The term olefin (B) in this text is thus to be interpreted in such a way that it does not relate to individual compounds but rather to mixtures of different olefins in which the desired isomerization product (olefin A) and other hydrocarbon compounds may also be present. However, the amounts of olefin (A) which are present in such mixtures are below the amount which is present in the thermodynamic equilibrium at the particular reaction temperature. Correspondingly, the same applies to the term olefin (A). This also includes mixtures of different olefins and hydrocarbons in which the olefin (B) serving as a starting material may also be present. This is in evidence from the fact alone that the double bond isomerization is an equilibrium reaction.

Preference is given to performing the process according to the invention in such a way that the olefin (A) is a 1-butenic C₄ hydrocarbon stream (1-C₄⁼ stream) and the olefin (B) used to prepare it is a 1-butenic and 2-butenic C₄ hydrocarbon stream (1- and 2-C₄⁼ feed stream) whose content of 1-butene is smaller than that at the thermodynamic equilibrium at the particular reaction temperature. It will be appreciated that the process can also be utilized to conversely convert 1-butene-rich C₄ hydrocarbon streams to those having a high 2-butene content.

The 1- and 2-C₄⁼ feed stream is C₄ cuts which generally have a content of butenes of from 30 to 100% by weight, preferably from 40 to 98% by weight, more preferably from 50 to 95% by weight. In addition to the butenes, the 1- and 2-C₄⁼ feed stream may also comprise up to 10% by weight, preferably up to 5% by weight, of polyunsaturated compounds or alkynes, in particular those having 3 or 4 carbon atoms such as butadienes, butynes, vinylacetylene, propyne and propadiene. In addition, from 0.5 to 60% by weight, preferably from 1 to 50% by weight, of C₄-alkanes and isobutene may be present. Further hydrocarbons having more than 5 carbon atoms, in particular pentanes and pentenes, are present, if appropriate, in amounts of up to a maximum of 10% by weight.

Especially suitable as 1- and 2-C₄⁼ feed streams are what are known as raffinates (raffinate I or II ).

Such raffinates I can be prepared by
subjecting naphtha or other hydrocarbon compounds to a steam cracking or FCC process and removing a C₄ hydrocarbon fraction from the stream formed
preparing from the C₄ hydrocarbon fraction a C₄ hydrocarbon stream (raffinate I) which consists substantially of isobutene, 1-butene, 2-butenes and butanes by hydrogenating the butadienes and butynes to butenes or butanes by means of selective hydrogenation, or removing the butadienes and butynes by extractive distillation.

Raffinates I are also obtainable by
preparing a C₄-olefin mixture from a hydrocarbon stream comprising butanes by dehydrogenating and subsequently isolating the C₄-olefin
preparing from the C₄-olefin mixture a C₄ hydrocarbon stream (raffinate I) which consists substantially of isobutene; 1-butene, 2-butenes and butanes by hydrogenating the butadienes and butynes to butenes or butanes by means of selective hydrogenation, or removing the butadienes and butynes by extractive distillation.

The raffinate II can be prepared from the raffinate I by removing the significant fraction of the isobutene from the raffinate I by known chemical, physicochemical or physical methods.

In a 3^{rd} method, raffinate II can be obtained by preparing a C₄-olefin mixture from methanol by dehydrogenation (MTO process) and if appropriate freeing it of butadienes or alkynes by distillation, partial hydrogenation or extractive distillation.

For further purification, the raffinate II may be freed of catalyst poisons by treating with adsorbant materials.

The compound (P) is preferably a compound having a dipole moment of from 0.5 to 5 debye, preferably from 0.75 to 4 debye, more preferably from 1 to 3 debye. So that the compound (P) may be in the gas phase under the reaction conditions, its boiling point at atmospheric pressure is generally below 200° C. Compounds having such properties are known to those skilled in the art. These are, for example, oxygen or nitrogen compounds, preferably C₁- to C₁₂-alkylamines, C₂- to C₆-alkylenediamines, such as ethyl-enediamine, cyclic amines in which 1 or 2 nitrogen atoms together with 1 or 2 alkanediyl groups form 5-, 6- or 7-membered rings, such as piperazine, triethylenediamine, C₁-to C12-alkyl alcohols, alkylene glycols, C₂- to C₁₂₋dialkyl ethers, cyclic ethers in which 1 or 2 oxygen atoms together with 1 or 2 alkanediyl groups form 5-, 6- or 7-membered rings, such as tetrahydrofuran or dioxane, water or ammonia. The definition of the compound (P) also comprises mixtures of compounds which have the dipole moment according to the definition.

The gaseous mixture which serves as a feedstock for the process according to the invention comprises from 0.01 to 10% by weight, preferably from 0.05 to 5% by weight, of the compound (P), based on the total amount of hydrocarbon compounds in this mixture.

Suitable for the process are basic catalysts, especially catalysts which comprise basic metal oxides. Preference is given to alkaline earth metal oxides, alkali metal oxides, alkaline earth metal aluminates or alkali metal aluminates. Particular preference is given to suitable catalysts comprising the elements sodium or potassium. Very particularly preferred catalysts are sodium aluminate, potassium aluminate, sodium oxide or potassium oxide, on gamma-aluminum oxide or a mixture of gamma-aluminum oxide and silicon dioxide.

Such catalysts are described, for example, in the following publications: EP 718036_A1 recommends the use of alkaline earth metal oxides supported on aluminum oxide. DE 3319171_A and DE 3319099_A disclose the use of oxides of the alkaline earth metals, boron group elements and lanthanides on mixed aluminum oxidelsilicon dioxide supports. The doping of magnesium-containing Al₂O₃ catalysts with alkali metal or zirconium is the subject matter of U.S. Pat. No. 4,889,840_A and U.S. Pat. No. 4,229,610_A. HU 204021_B mentions a method for preparing a catalyst by saturating aluminum oxide with an alkali metal compound and subsequently calcining. U.S. Pat. No. 4,229,610_A describes a catalyst consisting of aluminum oxide, sodium oxide and silicon dioxide. In Catalysis Surveys from Japan, Vol. 5, No. 2, April 2002, pages 81 ff, Yamaguchi et al. describe the double bond isomerization in olefins over alkali metal oxide catalysts on aluminum oxide or zirconium dioxide supports. To prepare the catalysts, the supports are initially impregnated with solutions of nitrates or carbonates of the alkali metals. Subsequently, the impregnated supports are heated to temperatures above the decomposition temperature of the nitrates or carbonates to form the alkali metal oxides.

The catalysts which are used in the process according to the invention are generally prepared by
a) impregnating a support comprising gamma-aluminum oxide with a solution of an alkali metal or alkaline earth metal nitrate, acetate, oxalate, oxide, hydroxide, hydrogencarbonate or carbonate (step a) and
b) drying the support saturated in step (a) and subsequently calcining it at a temperature of from 450 to 850° C.

The supports comprising gamma-aluminum oxide are commercially available and feature a surface area of from 100 to 400 m²/g and a pore volume of from 0.1 to 1.2 ml/g (measured by mercury porosimetry).

The solution with which the supports are impregnated in step (a) may also comprise mixtures of the salts mentioned.

The amount of solution of the aforementioned salts is such that if the assumption is made that the entire amount of the salts with which the supports are impregnated is converted in step (b) to the corresponding alkali metal or alkaline earth metal oxides, the weight of alkali metal or alkaline earth metal oxide, based on the total weight of the catalyst, is from 2 to 20% by weight, preferably from 5 to 15% by weight.

The catalysts are typically used in a fixed bed, fluidized bed or moving bed. In practical operation, it has been found that the amount of the 2-C₄⁼ stream which is passed over the catalyst per unit time is from 0.1 to 40 g (2-C₄⁼ stream)/[g (catalyst) h].

For the isomerization, preference is given to a continuous-flow fixed bed reactor system. Suitable reactors are tubular reactors, tube bundle reactors, tray reactors, coil reactors or helical reactors. The conversion of the 2-butenes to 1-butene is endothermic. The temperature control can be carried out as is customary. In addition, the reaction can also be performed in an adiabatic reaction system.

Olefin (B) may be in liquid or gaseous form. When olefin (B) is used in liquid form, it has to be evaporated before the reaction. The apparatus used for the evaporation is subject to no restriction. Suitable for this purpose are all customary evaporator types such as natural-circulation evaporators or forced-circulation evaporators. The gaseous olefin (B) stream is heated to reaction temperature in the apparatus which is customarily used, for example plate heat transferors or tube bundle heat transferors.

Compound P is added to the olefin (B) before the reaction. It may be metered in either in liquid or gaseous form. However, it has to be ensured that compound P is in gaseous form and at reaction temperature until it enters the reaction chamber. It is appropriate to evaporate and heat compound P together with the olefin (B).

The isomerization is carried out at a temperature at which shifting of the double bond is ensured, whereas cracking processes, skeletal isomerizations, dehydrogenations and oligomerizations are very substantially avoided. The reaction temperature is therefore generally from 200 to 700° C., preferably from 250 to 600° C., more preferably from 300 to 500° C. The pressure is adjusted in such a way that the olefin (B) is in gaseous form. The pressure is generally from 0.1 to 40 bar, preferably from 1 to 30 bar, more preferably from 3 to 20 bar.

The compound (P) is typically removed from the olefin (A). This is effected by customary separating methods. In a specific embodiment, the compound (P) removed may be recycled and added again to the olefin (B) before it enters the reaction zone.

In the case that the compound (P) is water, the removal may be effected in the condensed phase using a phase separator. In relatively small amounts, water can be separated from the olefin (A) by molecular sieve or a distillation of the azeotrope.

A 1-C₄⁼ stream prepared by the process according to the invention is suitable in particular for the preparation of 3-hexene by metathesis. To this end, the 1-C₄⁼ stream is contacted with a customary metathesis catalyst at a temperature of from 20 to 350° C. Such metathesis catalysts are common knowledge and are described, for example, in EP-A-1134271. These are generally compounds of a metal of transition group VIb, VIIb or VIII of the Periodic Table of the Elements.

When the 1-C₄⁼ stream comprises alkynes or polyunsaturated compounds, it is recommended to free the 1-C₄⁼ stream of the compounds by subjecting it, in the presence of a palladium-containing catalyst, to a selective hydrogenation in which there is virtually no conversion of 1-butene to 2-butenes. Such a selective hydrogenation avoiding isomerization can be achieved by contacting the 1-C4⁼ stream at from 40 to 60° C. and a partial hydrogen pressure of from 0.5 to 10⁶ pascal with a catalyst bed composed of a supported palladium catalyst. This type of hydrogenation is common knowledge and is described, for example, in the monograph Petrochemical Processes, Volume 1, Synthesis—Gas Derivatives and Major Hydrocarbons, A. Chauvel, G. Lefebvre, L. Castex, Institut Francais du Petrol Publications, 1989, Editions Technip, 27 Rue Ginoux, 75737 Paris, Cedex 15, on pages 208 and 209.

A 1-butene-rich C4 stream prepared by the above-described process can also be used as a starting material for a multitude of reactions. Examples include: dimerization, oligomerization, epoxidation, carbonylation and copolymerization with ethylene.

Particular preference is given to integrating the process according to the invention as the process step (b) into the process described in DE-A 10311139.5. This relates to a process for producing a 1-butene-containing C₄-hydrocarbon stream (1-C₄⁼ stream) from a 1-butene- and 2-butene-containing C₄-hydrocarbon stream (1- and 2-C₄⁼ feed stream) whose 1-butene content is lower than that of the 1-C₄⁼ stream, by
a) feeding the 1- and 2-C₄⁼ feed stream and a 1-butene- and 2-butene-containing C₄-hydrocarbon stream (1- and 2-C₄⁼ recycle stream) whose 1-butene content is lower than that of the 1-C₄⁼ stream and which has been produced by means of step (b) below into a distillation column and taking off the 1-C₄⁼ stream and a 2-butene-containing C₄-hydrocarbon stream (2-C₄⁼ stream) whose 1-butene content is lower than that of the 1- and 2-C₄⁼ feed stream and of the 1- and 2-C₄⁼ recycle stream from the distillation column (step a) and
b) producing the 1- and 2-C₄⁼ recycle stream from the 2-C₄⁼ stream by bringing the 2-C₄⁼ stream into contact with an isomerization catalyst which catalyzes the conversion of 2-butenes into 1-butene in a reaction zone (step b).

### EXPERIMENTAL SECTION

### Experiment 1

2-Butene from Linde was admixed with ammonia and the mixture was evaporated at 40° C. According to GC analysis, the volume ratio of butene to ammonia in the vapor was 1 to 0.012. A metered gas supply was used to pass 8 liters (STP)/h at atmospheric pressure into a preheater (250° C.) and subsequently into the reactor heated to 400° C. The reactor was a coil reactor (d=6 mm, I=10 cm) which was filled with 5 g of catalyst and was disposed in an electrically heated convection oven. The catalyst used was gamma-aluminum oxide which had been impregnated with potassium carbonate and calcined at 850° C., and had a potassium content of 5.4% by weight. The reactor effluent was passed through a GC with FID. This gave the compositions listed in Tab. 1 (data are in GC area %). The selectivity with respect to linear butenes over the entire observation time was >98%.

TABLE 1
Composition of the reaction effluent, ammonia as compound (P).

| Run time [h] | 1-Butene | cis-2-Butene | trans-2-Butene |
|---|---|---|---|
| 0 (Feed) | 0.2 | 72.3 | 27.2 |
| 11 | 25.1 | 37.1 | 37.2 |
| 41 | 25.6 | 36.1 | 37.8 |
| 71 | 25.3 | 38.4 | 35.7 |
| 100 | 25.6 | 35.9 | 38.0 |
| 130 | 25.3 | 34.8 | 39.3 |

### Experiment 2

2-Butene from Linde (60 g/h) and water (1.3 g/h) were evaporated at a pressure of 6 bar and 200° C. The mixture was preheated to reaction temperature (400° C.) and passed through a tubular reactor heated to 400° C. (d=10 mm, I=1 m, 30 g of catalyst). The catalyst used was gamma-aluminum oxide which had been impregnated with potassium carbonate and calcined at 850° C., and had a potassium content of 5.4% by weight. The reactor effluent was passed through a GC with FID. This gave the compositions listed in Tab. 2 (data are in GC area %). The selectivity with respect to linear butenes over the entire observation time was >98%.

TABLE 2
Composition of the reaction effluent, water as compound (P).

| Run time [h] | 1-Butene | cis-2-Butene | trans-2-Butene |
|---|---|---|---|
| 0 (Feed) | 0.2 | 72.3 | 27.2 |
| 9 | 25.9 | 39.7 | 33.6 |
| 39 | 26.0 | 39.9 | 33.2 |
| 75 | 25.7 | 39.9 | 33.5 |
| 101 | 26.0 | 39.4 | 33.8 |
| 138 | 25.9 | 38.9 | 34.7 |
| 190 | 26.3 | 41.6 | 31.3 |

### Comparative Experiment

The experiment was carried out in a similar manner to Example 1. 2-Butene from Linde is evaporated at 40° C. and 8 liters (STP)/h are passed over the catalyst at 400° C. The reactor effluent was passed through a GC with FID. This gave the compositions listed in Tab. 3 (data are in GC area %).

TABLE 3
Composition of the reaction effluent, without compound (P).

| Run time [h] | 1-Butene | cis-2-Butene | trans-2-Butene |
|---|---|---|---|
| 0 (Feed) | 0.2 | 72.3 | 27.2 |
| 11 | 25.9 | 31.8 | 41.8 |
| 40 | 26.0 | 31.8 | 41.7 |
| 74 | 26.4 | 33.4 | 39.8 |
| 99 | 25.7 | 41.7 | 32.2 |
| 130 | 21.0 | 50.4 | 28.1 |

1. A process for preparing a C₄- to C₁₂-olefin (olefin A) from another C₄- to C₁₂-olefin (olefin B), wherein the olefin (A) and the olefin (B) differ with respect to the position of a double bond, the process comprising:
providing a gaseous mixture comprising the olefin (B) and from 0.01 to 10% by weight, based on the total amount of hydrocarbon compounds in this mixture, of a compound having a dipole moment of from 0.5 to 5 debye (compound P); and
contacting the gaseous mixture with a basic catalyst at a temperature of from 200 to 700° C.,
wherein the basic catalyst is a sodium aluminate, a potassium aluminate, a sodium oxide, or a potassium oxide, on a gamma-aluminum oxide or on a mixture of gamma-aluminum oxide and a silicon dioxide.

2. The process according to claim 1, wherein the olefin (A) is a 1-butenic C₄ hydrocarbon stream (1-C₄⁼ stream) and the olefin (B) is a 1-butenic and 2-butenic C₄ hydrocarbon stream (1- and 2-C₄⁼ feed stream) in which the content of 1-butene is smaller than that of the 1-C₄⁼ stream.

3. The process according to claim 2, wherein a 1- and 2-C₄⁼ feed stream is used in which the ratio of 2-butene to 1-butene is from 6:1 to 0.1:1.

4. The process according to claim 2, wherein a 1- and 2-C₄⁼ feed stream is used which comprises a maximum of 5% by weight of polyunsaturated compounds or alkynes.

5. The process according to claim 2, wherein a 1- and 2-C₄⁼ feed stream is used in which the content of butenes is from 30 to 100% by weight.

6. The process according to claim 1, wherein compound (P) is an oxygen or a nitrogen compound.

7. The process according to claim 1, wherein the compound (P) is at least one of C₁- to C₁₂-alkylamines, C₂- to C₆-alkylenediamines, cyclic amines in which 1 or 2 nitrogen atoms together with 1 or 2 alkanediyl groups form 5-, 6- or 7-membered rings, C₁- to C₁₂-alkyl alcohols, alkylene glycols, C₂- to C₁₂-dialkyl ethers, cyclic ethers in which 1 or 2 oxygen atoms together with 1 or 2 alkanediyl groups form 5-, 6-, or 7-membered rings, a water or an ammonia.

8. The process according to claim 1, wherein the catalyst is used in which the weight of alkali metal or alkaline earth metal oxide, based on the total weight of the catalyst, is from 2 to 20% by weight.

9. The process according to claim 1, wherein a catalyst is used which is obtainable by
impregnating a support comprising a gamma-aluminum oxide with a solution of an alkali metal or an alkaline earth metal nitrate, an acetate, an oxalate, an oxide, a hydroxide, a hydrogencarbonate or a carbonate; and
drying the support; and
subsequently calcining the support at a temperature of from 450 to 850° C.
